(19) United States
(12) Patent Application Publication
(10) Pub. No.: US 2023/0416373 A1
(43) Pub. Date: Dec. 28, 2023

(54) BIPHASIC SUBCUTANEOUS DOSING REGIMENS FOR ANTI-VLA-4 ANTIBODIES

(71) Applicant: BIOGEN MA INC., Cambridge, MA (US)

(72) Inventors: Yuan ZHAO, Cambridge, MA (US); Himanshu NAIK, Cambridge, MA (US); Nolan CAMPBELL, Cambridge, MA (US)

(21) Appl. No.: 18/036,620

(22) PCT Filed: Nov. 12, 2021
(86) PCT No.: PCT/US2021/059266

§ 371 Date: May 11, 2023

Related U.S. Application Data
(60) Provisional application No. 63/113,864, filed on Nov. 14, 2020.
(60) Provisional application No. 63/142,968, filed on Jan. 28, 2021.

(51) Int. Cl. C07K 16/28; A61P 25/00; A61K 9/00

(52) U.S. Cl. CPC C07K 16/2842; A61P 25/00; A61K 9/0019; A61K 2039/505

(57) ABSTRACT

Provided herein are biphasic dosing protocols for natalizumab therapy comprising both standard and extended interval dosing and subcutaneous administration.

### RELATED APPLICATIONS

This application claims the benefit of priority to U.S. Provisional Patent Application Ser. No. 63/113,864, filed Nov. 14, 2020 and to U.S. Provisional Patent Application Ser. No. 63/142,968, filed Jan. 28, 2021, each of which is incorporated by reference herein for all purposes.

### BACKGROUND

Subcutaneous (SC) delivery of biotherapeutics is a valued alternative to intravenous (IV) administration and can significantly reduce the treatment burden for patients, including less time spent in the clinic, greater comfort during administration, less emotional distress while receiving treatment, and lower levels of injection site pain. Rummel et al., Ann. Oncol. 2017; 28(4):836.842. In addition, the convenience and speed of SC administration can lead to time and cost savings in the health care system, relieve strain on infusion centers and allow greater patient access. Dychter et al., J. Infus Nurs. 2012; 35(3):154.160.

Unfortunately, however, the pharmacokinetic profiles between the two routes of administration can differ significantly (Bittner et al., Subcutaneous administration of biotherapeutics: an overview of current challenges and opportunities. BioDrugs. 32:425.440, 2018). Intravenous administration of a biotherapeutic results in an almost immediate maximum serum concentration (Cₘₐₓ), whereas SC administration is typically slowly adsorbed resulting in Cₘₐₓ levels well below that obtained with IV dosing (Id., page 432). This is a consequence of the limited permeability of biotherapeutics across the vascular endothelia, as well as interactions with interstitial glycosaminoglycans and proteins, and enzymatic degradation. Incomplete bioavailability is thus a hallmark of SC in comparison with IV administration, such that increased SC dosing is often needed to achieve an equivalent therapeutically effective concentration. Because pharmacodynamics and pharmacokinetics between the two routes of administration differ, and are specific to each individual biotherapeutic (Id., page 436), caution and uncertainty remain for the use of SC administration for a biotherapeutic which is traditionally delivered by IV administration. As but one more recent example, Cinquair® (reslizumab) is an approved IV treatment for asthma but failed as a fixed-dose subcutaneous treatment in several phase 3 clinical trials. Bernstein et al. Lancet Respir Med. doi:10.1016/S2213.2600(19)30372.8 (February 2020).

Tysabri® (natalizumab) is an anti-very late antigen (VLA)-4 humanized monoclonal IgG4 antibody that inhibits the migration of lymphocytes throughout the blood-brain barrier by blocking VLA-4 interactions with vascular cell adhesion molecules (VCAM)-1 and reducing inflammatory lesions. Natalizumab is a biotherapeutic approved for treating multiple sclerosis. Progressive multifocal leukoencephalopathy (PML), an opportunistic infection caused by the John Cunningham virus (JCV) that only occurs in patients who are immunocompromised, has affected a small population of patients using natalizumab.

Previously, use of an extended interval IV dosing regimen, in order to minimize PML risk, has been disclosed (WO 2019/085335). Subcutaneous formulations of VLA-4 binding antibody, for monthly or bi-monthly administration, have also been previously been disclosed (U.S. Pat. No. 9,533,044). However, the two therapeutic approaches follow dissimilar routes and duration of administration.

Thus, there remains a need for a reliable biotherapeutic regimen that reduces pathological inflammation while still delivering a therapeutically effective concentration of the biotherapeutic.

### SUMMARY

The present disclosure provides improved biphasic dosing regimens for reducing pathological inflammation with anti-VLA-4 antibodies, and with natalizumab in particular, wherein the dosing regimens comprise an induction phase employing standard interval dosing (SID) followed by a chronic phase employing extended interval dosing (EID). In some embodiments, the same dose administered during the SID phase can be administered during the EID phase. In preferred embodiments, at least one treatment phase and more preferably both treatment phases employ subcutaneous administration. In particular, and as detailed herein, the present inventors have surprisingly determined that subcutaneous (SC) administration can be substituted for intravenous (IV) administration in one or both phases of the dosing regimen, leading to substantial improvements in safety, cost and patient compliance. Surprisingly, the trough natalizumab concentration and alpha-4 integrin saturation are similar for IV and SC administration, regardless of patient body weight. In some embodiments, therefore, the same dose administered IV can be administered SC, thus greatly simplifying clinical implementation and supply chain logistics. Last but not least, the improved regimens and methods provided herein also further reduce the risk of developing PML without substantially compromising efficacy.

Provided are methods for chronically reducing pathological inflammation in a patient in need thereof, such methods comprising administering to said patient a therapeutically effective amount of an anti-VLA-4 antibody in a biphasic dosing regimen, wherein the biphasic regimen comprises an induction phase comprising administration of the anti-VLA-4 antibody once every 2 weeks, about once every 2 weeks, once every 4 weeks, about once every 4 weeks, once every 30 days, about once every 30 days, once a month or about once a month for at least 6 months, more preferably for at least 8, 10 or 12 months, followed by a chronic phase comprising administration of the anti-VLA-4 antibody once every 5 to 10 weeks, more preferably once every 5, 6, 7 or 8 weeks. In some embodiments, the induction phase is from 6 to 18 months, from 8 to 16 months, from 10 to 14 months, is 11 months, is 12 months, or is 13 months. In particular embodiments, the induction phase is 12 months, and the chronic phase comprises administration of the anti-VLA-4 antibody every 5 weeks, about every 5 weeks, every 6 weeks, about every 6 weeks, 7 weeks or about every 7 weeks, more preferably every 6 weeks or about every 6 weeks.

In some embodiments, the anti-VLA-4 antibody is natalizumab. In some embodiments, the SC dosing and amount can be consistent with IV dosing. In some embodiments, the therapeutically effective amount administered during the induction phase and the chronic phase are the same, and the therapeutically effective amount is between 250-450 mg (e.g., 250 mg, 300 mg, 350 mg, 400 mg, or 450 mg), more preferably about 300 mg, still more preferably 300 mg. In some embodiments, the therapeutically effective amount administered SC during the chronic phase is between 300.500 mg (e.g., 300 mg, 350 mg, 400 mg, 450 mg, or 500 mg). In some embodiments, the therapeutically effective amount is between about 250-about 450 mg (e.g., about 250 mg, about 300 mg, about 350 mg, about 400 mg, or about 450 mg), more preferably about 300 mg, still more preferably 300 mg. In some embodiments, the therapeutically effective amount administered SC during the chronic phase is between about 300-about 500 mg (e.g., about 300 mg, about 350 mg, about 400 mg, about 450 mg, or about 500 mg), more preferably about 300 mg, still more preferably 300 mg.

In preferred embodiments, at least one treatment phase and preferably both treatment phases comprise SC administration, and preferably solely SC administration. In some embodiments, the induction phase comprises SC administration, and preferably solely SC administration. In some embodiments, the chronic phase comprises SC administration, and solely SC administration. In some embodiments, both the induction phase and the chronic phase comprise SC administration, and preferably solely SC administration. In exemplary embodiments, the patient is naïve to natalizumab therapy, and the pathological inflammation is multiple sclerosis.

Additional aspects of the present disclosure provide methods of reducing risk of developing progressive multifocal leukemia (PML) in a subject having multiple sclerosis, comprising administering to said patient a therapeutically effective amount of an anti-VLA-4 antibody in a biphasic dosing regimen, wherein the biphasic regimen comprises an induction phase comprising administration of the anti-VLA-4 antibody once every 2 weeks, about once every 2 weeks, once every 4 weeks, about once every 4 weeks, once a month or about once a month for at least 6 months, more preferably for at least 8, 10 or 12 months, followed by a chronic phase comprising administration of the anti-VLA-4 antibody once every 5 to 10 weeks, more preferably once every 5, 6, 7 or 8 weeks. In some embodiments, the induction period is from 6 to 18 months, from 8 to 16 months, from 10 to 14 months, is 11 months, is 12 months, or is 13 months. In particular embodiments, the induction phase is 12 months, and the chronic phase comprises administration of the anti-VLA-4 antibody every 5 weeks, about every 5 weeks, every 6 weeks, about every 6 weeks, every 7 weeks, or about every 7 weeks, more preferably every 6 weeks or about every 6 weeks.

In some embodiments, the anti-VLA-4 antibody is natalizumab. In some embodiments, the SC dosing and amount can be consistent with IV dosing. In some embodiments, the therapeutically effective amount administered during the induction phase and the chronic phase are the same, and the therapeutically effective amount is between 250-450 mg (e.g., 250 mg, 300 mg, 350 mg, 400 mg, or 450 mg), more preferably about 300 mg, still more preferably 300 mg. In some embodiments, the therapeutically effective amount administered SC during the chronic phase is between 300.500 mg (e.g., 300 mg, 350 mg, 400 mg, 450 mg, or 500 mg. In some embodiments, the therapeutically effective amount is between about 250-about 450 mg (e.g., about 250 mg, about 300 mg, about 350 mg, about 400 mg, or about 450 mg), more preferably about 300 mg, still more preferably 300 mg. In some embodiments, the therapeutically effective amount administered SC during the chronic phase is between about 300-about 500 mg (e.g., about 300 mg, about 350 mg, about 400 mg, about 450 mg, or about 500 mg), more preferably about 300 mg, still more preferably 300 mg

In preferred embodiments, at least one treatment phase and preferably both treatment phases comprise SC administration. In some embodiments, the induction phase comprises SC administration, and preferably solely SC administration. In some embodiments, the chronic phase comprises SC administration, and solely SC administration. In some embodiments, both the induction phase and the chronic phase comprise SC administration, and preferably solely SC administration. In exemplary embodiments, the patient is naïve to natalizumab therapy, and the pathological inflammation is multiple sclerosis.

In another aspect, the present invention provides a method of administering natalizumab to a patient in need thereof based on a biphasic treatment protocol, the method comprising administering the natalizumab therapy on an SID schedule of 4-week intervals for an induction phase of at least 12 months, and then administering the natalizumab therapy on an EID schedule of at least 6-week intervals chronically thereafter, wherein one or both, and preferably both, treatment phases comprise SC administration, and still more preferably solely SC administration. In certain embodiments, the chronic administration phase may be from 6 months to 48 months, from 8 months to 36 months, from 12 months to 24 months, or in other embodiments greater than 4 years, greater than 8 years or for the life of the patient.

In some embodiments, the subject methods further comprise a) measuring a soluble molecule in a first biological sample obtained from the patient during the induction phase, wherein the soluble molecule is sVCAM; b) measuring the sVCAM in a second biological sample obtained from the individual during the chronic phase; c) determining whether there is an increase in the levels of the sVCAM within an acceptable range between the first and second biological samples, e.g. between about >0 and about <600 ng/mL, wherein the range correlates with an acceptable increase in immune surveillance activity in the individual, and d) in the event of an unacceptable increase in the levels of sVCAM, e.g., >600 ng/mL, reverting said patient to a SID schedule of 4-week intervals and/or a once-a-month dosing regimen, or increasing the dose frequency of the EID schedule, e.g. administering every five weeks instead of every six weeks. In some embodiments, the first biological sample is obtained from the patient at least 6 months after initiation of the induction phase, and the second biological sample is obtained within 6 months of the initiation of the chronic phase.

In some embodiments, the subject methods further comprise a) measuring a soluble molecule in at least one first biological sample obtained from the patient during the induction phase, wherein the soluble molecule is neurofilament light chain (Nf-L); b) measuring the Nf-L in at least one second biological sample obtained from the individual during the chronic phase; c) determining whether there is an increase in the levels of Nf-L between the first and second biological samples, wherein the increase correlates with ongoing neuronal damage in the individual, and d) in the event of an increase reverting said patient to a SID schedule of 4-week intervals and/or a once-a-month dosing regimen, or increasing the dose frequency of the EID schedule, e.g. administering every five weeks instead of six weeks. In some embodiments, the first biological sample is obtained from the patient at least 6 months after initiation of the induction phase, and the second biological sample is obtained within 6 months of the initiation of the chronic phase.

Other aspects of the present disclosure provide methods of improving the efficacy of chronic natalizumab therapy, comprising administering to a subject a therapeutically effective amount of natalizumab on a SID schedule of 4-week intervals for at least 12 months, followed by chronically administering to the subject a therapeutically effective amount of natalizumab on an EID schedule of at least 6-week intervals, monitoring whether the mean trough α4-integrin receptor saturation is greater than 40, 50, 60, or 70% in the patient, and reverting the patient back to an SID schedule if the α4-integrin receptor saturation falls below the requisite threshold, wherein one or both, and preferably both the SID and the EID schedules comprise SC administration. In some embodiments, the SID and/or EID schedules comprise a dose of 300 milligrams, and preferably both.

In some embodiments, monitoring the mean trough α4-integrin receptor saturation comprises a) measuring a soluble molecule in a biological sample obtained from the patient during the SID schedule, wherein the soluble molecule is sVCAM; b) measuring the sVCAM in a second biological sample obtained from the individual during the EID schedule; c) determining whether there is an increase in the levels of the sVCAM between the first and second biological samples, wherein the increase correlates with an increase in alpha-4 integrin activity in the individual, and d) in the event of an increase above a threshold amount, e.g., >600 ng/mL, reverting said patient to an SID schedule, or increasing the dose frequency of the EID schedule, e.g. administering every five weeks instead of six weeks. In some embodiments, the first biological sample is obtained from the patient at least 6 months after initiation of the SID schedule, and the second biological sample is obtained within 6 months of the initiation of the EID schedule.

In some embodiments, the subject has an autoimmune disease. In some embodiments, the autoimmune disease is MS. In some embodiments, the autoimmune disease is an inflammatory bowel disease. In some embodiments, the autoimmune disease is Crohn's disease. In some embodiments, the subject is diagnosed with, or has, epilepsy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Distribution of simulated natalizumab steady-state PD/PK parameters. Boxes indicate the interquartile range, and the whiskers indicate the range excluding the outliers. Open circles represent outliers that are > or <1.5 times the value of first or third quartile. The horizontal line within each box represents the median. C_{avg} is defined as AUCₜₐᵤ/tau, with tau being 672 h. C_{trough} is the minimum natalizumab serum concentration or α4-integrin saturation.

FIG. 2: PK profile of natalizumab following IV and SC treatment. Arrows indicate the time of dosing.

FIG. 3: alpha-4 integrin saturation levels over time following natalizumab IV and SC treatment. Arrows indicate the time of dosing.

FIG. 4: Soluble VCAM levels over time following natalizumab IV and SC treatment. Arrows indicate the time of dosing.

FIG. 5A-B: Pharmacokinetic profiles following different dose regimens: Q4W or Q6W (following 12-month Q4W natalizumab treatment). At baseline the mean trough concentrations were similar for both Q6W and Q4W groups (Q6W: 34.0929 μg/mL; Q4W: 34.2811 μg/mL). Mean trough concentrations following Q6W and Q4W dosing interval ranged from 10 μg/mL to 21 μg/mL for Q6W, and 33 μg/mL to 38 μg/mL for Q4W. In general, mean trough concentrations following Q6W dosing interval were approximately 60% to 70% lower than those observed for the Q4W dosing interval. Administration was by IV.

FIG. 6A-B: Pharmacodynamic profiles following different dose regimens: Q4W or Q6W (following 12-month Q4W natalizumab treatment). Observations <10% or >150% are not shown on plots (n=4 for Q4W and n=22 for Q4W to Q6W). At baseline the mean trough α4 integrin saturation on MNCs was similar for both Q4W and Q6W treatment groups. For the Q6W dosing interval, α4 integrin saturation declined by 8% to 12% at Week 12 and then remained stable above 63% during the study. For the Q4W dosing interval, α4 integrin saturation consistently maintained above 76% throughout the study. Compared with the Q4W group, α4 integrin saturation was 9% to 16% lower across the timepoints in the Q6W group.

FIG. 7: IV vs SC natalizumab steady state C_{trough} following administration of natalizumab 300 mg Q6W. The upper and lower boundaries of the box represent the 75^{th} and 25^{th} percentiles, respectively. The solid line within the box marks the median. The whisker bars indicate the 97.5^{th} and 2.5^{th} percentiles of the simulation results.

FIG. 8: IV vs SC alpha-4-integrin saturation at steady state C_{trough} following administration of natalizumab 300 mg Q6W. The upper and lower boundaries of the box represent the 75^{th} and 25^{th} percentiles, respectively. The solid line within the box marks the median. The whisker bars indicate the 97.5^{th} and 2.5^{th} percentiles of the simulation results.

FIG. 9A-B: Predicted steady-state trough (A) natalizumab concentration and (B) alpha-integrin saturation after SC 300 mg Q4W dosing by body weight.

FIG. 10A-B: Predicted steady-state trough (A) natalizumab concentration and (B) alpha-integrin saturation after SC 300 mg Q4W dosing by prior natalizumab exposure.

FIG. 11A-B: Model predicted (A) steady-state trough pharmacokinetic (natalizumab concentration) and (B) pharmacodynamic (alpha-4 integrin saturation) endpoints by body weight category. The predicted steady-state Q6W natalizumab concentration and alpha-4 integrin saturation decreased with increasing body weight similarly with intravenous and subcutaneous administration.

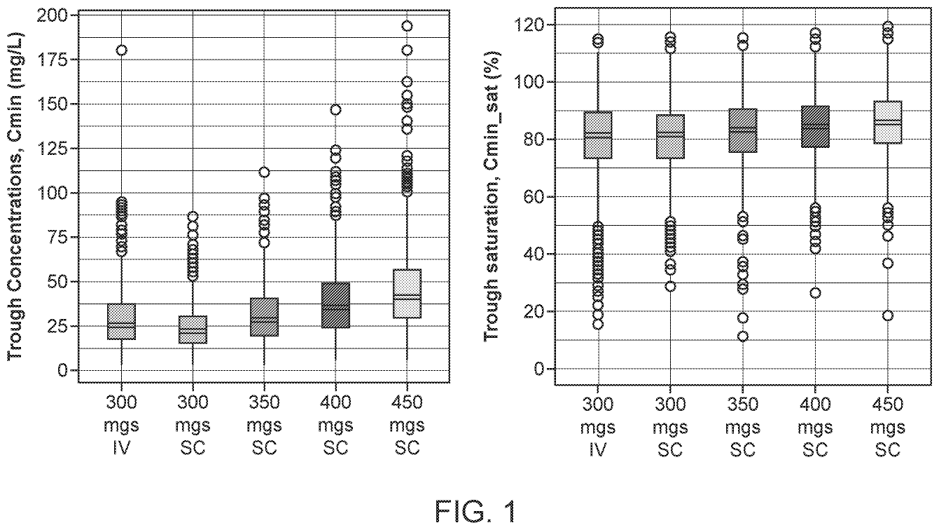

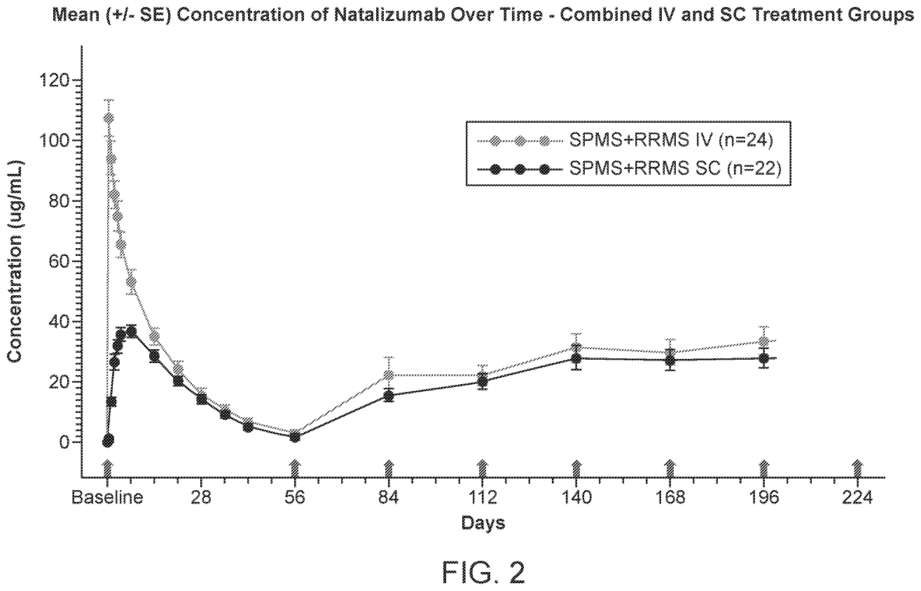

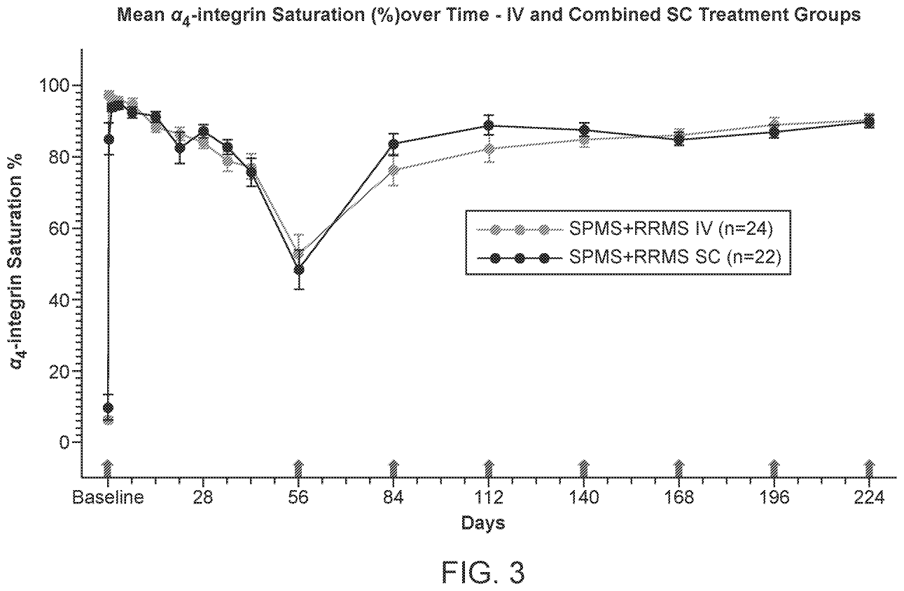

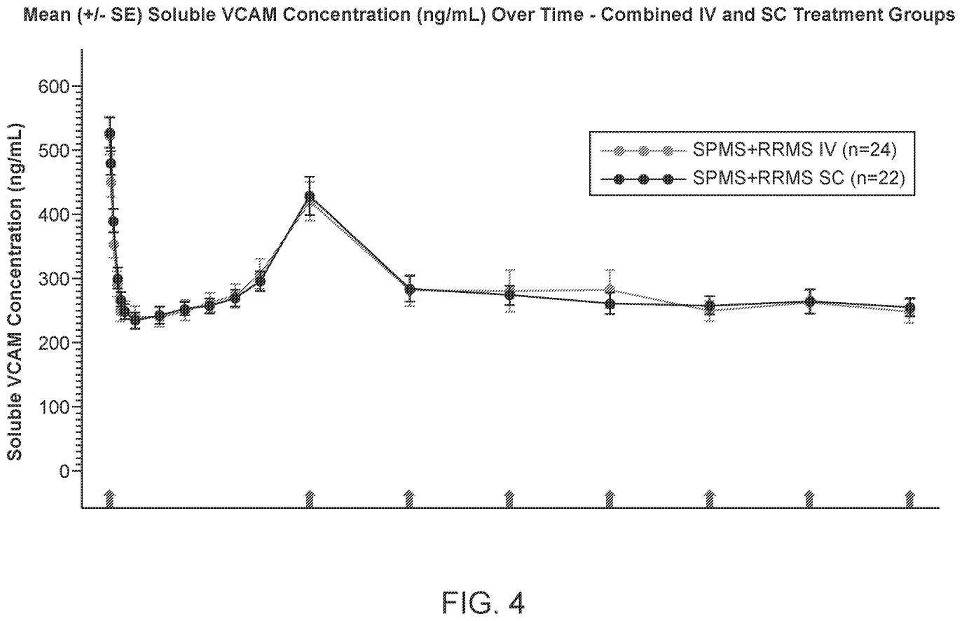

### DETAILED DESCRIPTION

Natalizumab, sold under the trade name TYSABRI® (BIOGEN®, MA), is an integrin receptor antagonist approved by the U.S. Food and Drug administration (FDA) for treatment of multiple sclerosis and Crohn's disease. The FDA approved standard dosing regimen is 300 milligrams (mg) infused intravenously over approximately one hour, every four weeks. Among the population of patients who have received natalizumab therapy, there is a small subpopulation of patients who have developed progressive multifocal leukoencephalopathy (PML) (Plavina, T. et al. Ann Neurol 2014; 76:802.12). Substantial efforts have been made to identify and minimize this risk, including the development a wide range of patient monitoring and alternative dosing protocols. Nevertheless, it has proven difficult to balance increasing the safety of natalizumab treatment without concomitantly decreasing its efficacy, because natalizumab's risk and efficacy are likely mechanistically related.

### Definitions

A subject, as provided herein, is typically a male or female human subject (patient) who is undergoing or who will undergo treatment with natalizumab for a particular condition. The condition may be an autoimmune condition or an inflammatory condition. Often, autoimmune conditions are considered inflammatory conditions and vice versa, thus, in some embodiments the subject has an autoimmune condition and/or inflammatory condition. An autoimmune condition is a condition in which a subject's immune system attacks the subject's own cells/tissues. Non-limiting examples of autoimmune conditions include multiple sclerosis (MS) (e.g., relapsing-remitting MS, secondary progressive MS, and/or primary progressive MS), Crohn's disease, rheumatoid arthritis, lupus, celiac disease, Sjorgren's syndrome, Polymyalgia rheumatic, ankylosing spondylitis, Type 1 diabetes, alopecia areata, vasculitis, and temporal arteritis.

As used herein, “about” refers to within 0.1% to 5% of the given value (e.g., within 5%, 3%, 2%,1%, 0.5%, 0.1% above or below the given value). Where amounts and other designated values are provided herein, the allowable deviation is within pharmaceutically acceptable standards.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein. A “pharmaceutically effective amount” or “therapeutically effective amount,” used interchangeably, is an amount sufficient to cure or at least partially arrest the symptoms of a disease and/or the complications of a disease.

An “anti-VLA-4 antibody” is an anti-very late antigen (VLA)-4 monoclonal antibody, a humanized, a human, or a chimeric anti-VLA-4 monoclonal antibody.

A biphasic dosing regimen herein refers to the administration of natalizumab in at least two phases, e.g., an induction phase and a chronic phase. Preferably, the induction phase comprises administration of natalizumab on an SID schedule and the chronic phase comprises administration of natalizumab on an EID schedule. In some embodiments, the induction phase comprises administration of natalizumab once every 2 weeks, about once every 2 weeks, once every 3 weeks, about once every 3 weeks, once every 4 weeks, about once every 4 weeks, once every 30 days, about once every 30 days, once a month or about once a month for at least 6 months, more preferably for at least 8, 10 or 12 months. In some embodiments, the induction phase is from 6 to 18 months, from 8 to 16 months, from 10 to 14 months, is 11 months, is 12 months, or is 13 months. In some embodiments, the chronic phase comprises administration of natalizumab once every 5 to 10 weeks, more preferably once every 5 weeks, about every 5 weeks, every 6 weeks, about every 6 weeks, every 7 weeks, about every 7 weeks, every 8 weeks, or about every 8 weeks. In particular embodiments, both the induction phase and the chronic phase comprise SC administration, and preferably solely SC administration.

Extended interval dosing (EID) herein refers to the administration of natalizumab at intervals that extend beyond the standard interval dosing (SID) dosing schedule of 300 mg every 4 weeks. An EID schedule should not exceed 12 doses of natalizumab within a 12-month period (one month equals 30 days), and typically does not exceed 11 or 10 doses within a 12-month period (one month equals 30 days). Thus, a SID schedule should exceed 10 doses of natalizumab within a 12-month period, and typically exceeds 11 or 12 doses in a 12-month period. In some embodiments, the EID schedule interval for administering natalizumab (e.g., 300 mg dose) is at least 5 weeks (35 days). For example, the EID schedule interval may be at least 6 weeks, at least 7 weeks, at least 8 weeks, at least 9 weeks, or at least 10 weeks. In some embodiments, the EID schedule interval is 5.12 weeks. For example, the EID schedule interval may be 5.10 weeks, 5.9 weeks, 5.8 weeks, 5.7 weeks, or 5.6 weeks. In some embodiments, the EID schedule interval is about 5 weeks, about 6 weeks, about 7 weeks, about 8 weeks, about 9 weeks, or about 10 weeks. In some embodiments, the EID schedule interval is 5 weeks, 6 weeks, 7 weeks, 8 weeks, 9 weeks, or 10 weeks. In some embodiments, the EID schedule interval is 5 weeks and 1 day, 5 weeks and 2 days, 5 weeks and 3 days, 5 weeks and 4 days, 5 weeks and 5 days, or 5 weeks and 6 days. In some embodiments, the EID schedule interval is 6 weeks and 1 day, 6 weeks and 2 days, 6 weeks and 3 days, 6 weeks and 4 days, 6 weeks and 5 days, or 6 weeks and 6 days. In some embodiments, the EID schedule is 7 weeks and 1 day, 7 weeks and 2 days, 7 weeks and 3 days, 7 weeks and 4 days. 7 weeks and 5 days, or 7 weeks and 6 days. In some embodiments, the EID schedule interval is 8 weeks and 1 day, 8 weeks and 2 days, 8 weeks and 3 days, 8 weeks and 4 days, 8 weeks and 5 days, or 8 weeks and 6 days. In some embodiments, the EID schedule interval is 9 weeks and 1 day, 9 weeks and 2 days, 9 weeks and 3 days, 9 weeks and 4 days, 9 weeks and 5 days, or 9 weeks and 6 days.

In some embodiments, the interval of the EID schedule and/or chronic phase of the dosing regimen is greater than 4 weeks and less than 10 weeks, or greater than about 4 weeks and less than about 10 weeks. In some embodiments, the interval of the EID schedule and/or chronic phase of the dosing regimen is at least 5 weeks and less than 10 weeks, or at least about 5 weeks and less than about 10 weeks. In some embodiments, the interval of the EID schedule and/or chronic phase of the dosing regimen is greater than 5 weeks or about 5 weeks and no more than 6, 7 or 8 weeks or about 6, 7, or 8 weeks. In some embodiments, the interval of the EID schedule and/or chronic phase of the dosing regimen is at least about 6 weeks and no more than about 9 weeks. In some embodiments, the interval of the EID schedule and/or chronic phase of the dosing regimen is greater than 5 weeks and no more than 8 weeks, or greater than about 5 weeks and no more than about 8 weeks. In some embodiments, the interval of the EID schedule and/or chronic phase of the dosing regimen is at least 6 weeks and no more than 8 weeks, or at least about 6 weeks and no more than about 8 weeks. In certain embodiments, the chronic administration period may be 6 months to 48 months, about 6 months to about 48 months, from 8 months to 36 months, from about 8 months to about 36 months, from 12 months to 24 months, from about 12 months to about 24 months, or in other embodiments greater than 4 years, greater than about 4 years, greater than 8 years, greater than about 8 years, or for the life of the patient.

In some embodiments, the biphasic dosing regimen is selected to maintain a mean trough α4β1-integrin receptor saturation of greater than 40%, 45% or 50% (or greater than 55%, 60%, 65%, or 70%), or at least about 40%, 45% or 50% (or at least about 55%, 60%, 65%, or 70%), during both treatment phases in a population of patients in need of natalizumab treatment. In some cases, the biphasic dosing regimen that maintains a referenced (greater than 40%, 45%, 50%, 55%, 60%, 65%, or 70%, or at least about 40%, 45%, 50%, 55%, 60%, 65%, or 70%) mean trough α4β1-integrin receptor saturation in a population of patients in need of natalizumab treatment is an SID schedule for at least 12 months, followed by chronic administration on an EID schedule having an interval of from greater than 5 weeks to no more than 10 weeks. In some cases, the EID schedule interval that maintains a referenced mean trough α4β1-integrin receptor saturation during the chronic phase in a population of patients in need of natalizumab treatment is an EID schedule having an interval of from at least 6 weeks, from greater than 4 weeks to less than 10 weeks, from greater than 5 weeks to no more than 10 weeks, from at least 6 weeks to less than 10 weeks, or from at least 6 weeks to no more than 7, 8 or 9 weeks.

In some cases, the biphasic dosing regimen comprises an SID schedule comprising administration of natalizumab once every 2 weeks, about once every 2 weeks, once every 4 weeks, about once every 4 weeks, once every 30 days, about once every 30 days, once a month, or about once a month for at least 6 months, more preferably for at least 8, 10 or 12 months, followed by an EID schedule comprising administration of natalizumab once every 5 to 10 weeks, more preferably once every 5, 6, 7 or 8 weeks. In some embodiments, the SID schedule is from 6 to 18 months, from 8 to 16 months, from 10 to 14 months, is 11 months, is 12 months, or is 13 months. In particular embodiments, the SID schedule is 12 months, and the EID schedule comprises administration of natalizumab every 5 weeks, about every 5 weeks, every 6 weeks, about every 6 weeks, every 7 weeks, or about every 7 weeks, more preferably every 6 weeks, or about every 6 weeks.

In some embodiments, an EID schedule includes 15 doses or fewer over an 18-month period. In other embodiments, an EID schedule includes 10 doses or fewer over a 12-month period. In some embodiments, an EID schedule includes 10 doses or fewer per year over the duration of infusion history. In some embodiments, an EID schedule includes at least 5 doses during the chronic phase. In some embodiments, an EID schedule includes at least 6 doses during the chronic phase. In some embodiments, an EID schedule includes 5 doses or more during the chronic phase. In some embodiments, an EID schedule includes 6 doses or more during the chronic phase.

In some embodiments, an EID schedule is followed (is administered) over the course of at least 6 months. In some embodiments, an EID schedule is followed over the course of at least 12 months (1 year). In some embodiments, an EID schedule is followed over the course of at least 18 months. In some embodiments, an EID schedule is followed over the course of at least 24 months (2 years). In some embodiments, an EID schedule is followed over the course of at least 30 months. In some embodiments, an EID schedule is followed over the course of at least 36 months (3 years). In some embodiments, an EID schedule is followed over the course of at least 48 months (4 years). In some embodiments, an EID schedule is followed over the course of at least 96 months (8 years). In some embodiments, an EID schedule is followed over the course of the patient's lifetime.

In some embodiments, an SID schedule includes natalizumab 300 mg SC administration every 4 weeks (−2/+5 days), e.g., up to Week 52. In some embodiments, an EID schedule includes natalizumab 300 mg SC administration every 6 weeks (−2/+5 days), e.g., after Week 52. In some embodiments, an EID schedule includes 350 mg, 400 mg, 450 mg, or 500 mg natalizumab SC administration every 6 weeks (−2/+5 days), e.g., after Week 52. In some embodiments, an EID schedule includes 150 mg, 175 mg, 200 mg, 225 mg, or 250 mg natalizumab SC administration every 3 weeks (−2/+5 days), e.g., after Week 52.

In some embodiments, monitoring the mean trough (4-integrin receptor saturation comprises a) measuring a soluble molecule in a first biological sample obtained from the patient during the SID schedule, wherein the soluble molecule is sVCAM; b) measuring the sVCAM in a second biological sample obtained from the individual during the EID schedule; c) determining whether there is an increase in the levels of sVCAM between the first and second biological samples, wherein the increase correlates with an increase in (4-integrin activity in the individual, and d) in the event of an increase above a threshold amount, e.g., >600 ng/mL, reverting said patient to an SID schedule, or increasing the dose frequency of the EID schedule, e.g. administering every five weeks instead of six weeks. In some embodiments, the first biological sample is obtained from the patient at least 6 months after initiation of the SID schedule, and the second biological sample is obtained within 6 months of the initiation of the EID schedule.

Relevant biomarkers for determining and/or monitoring efficacy of the biphasic treatment protocols provided herein include, e.g., sVCAM and/or Nf-L. Without being bound by theory, increased saturation and/or occupancy by natalizumab of its target α4 integrin on the surface of circulating lymphocytes leads to decreased surface expression of α4-integrin on lymphocytes, as well as decreased serum concentration of sVCAM. Correspondingly, sVCAM provides an effective surrogate biomarker for α4-integrin receptor saturation, and for immune surveillance activity in general, see, e.g. Plavina et al., Neurology (2017) 89(15):1584.1593. Neurofilament proteins such as Nf-L, in contrast, provide an indication of axonal damage and neuronal death, and serve as effective surrogate biomarkers for ongoing disease activity in MS patients in particular. See, e.g., Kuhle et al. Mult Scler. (2013) 19:1597.603; Varhaug et al., Front Neurol. (2019) 10: 338.

In alternative embodiments, the subject methods further comprise determining and/or monitoring the efficacy of the biphasic dosing protocols disclosed herein, comprising a) measuring a soluble molecule in a first biological sample obtained from the patient during the induction phase, wherein the soluble molecule is sVCAM and/or Nf-L; b) measuring the sVCAM and/or Nf-L in a second biological sample obtained from the individual during the chronic phase; c) determining whether there is an increase in the levels of the sVCAM and/or Nf-L above predetermined thresholds between the first and second samples, and d) in the event of an increase above one or both predetermined thresholds reverting said patient to a SID schedule of 4-week intervals and/or a once-a-month dosing regimen, or increasing the dose frequency of the EID schedule, e.g. administering every five weeks instead of every six weeks. In some embodiments, the first biological sample is obtained from the patient at least 6 months after initiation of the induction phase, and the second biological sample is obtained within 6 months of the initiation of the chronic phase.

In some embodiments, the soluble marker is sVCAM, and the predetermined threshold is >600 ng/mL. In some embodiments, the soluble marker is Nf-L, and the predetermined threshold is any increase greater than the test-retest variability in the assay, whether on a per-patient level or on a population level. In some embodiments, the soluble marker is Nf-L, and step c) comprises determining whether there is an increase in the second sample over an on-treatment or steady state value established by the first sample for the individual patient, or an established on-treatment value derived from the literature. See, e.g., Delcoigne et al., Neurology (2020) 94:e1201-e1212; Kuhle et al. J. Multiple Sclerosis (2020) 26(13):1691.99.

Treatment with natalizumab is also associated with increases in circulating immune cells from prenatalizumab levels, and CD34+ hematopoietic progenitor cells in particular have been found to increase above normal reference levels. Polman et al., N. Engl J. Med (2006) 354:899.910; Bonig et al., Blood (2008) 111:3439.3441. In additional embodiments, therefore, the subject methods further comprise determining the efficacy of the biphasic dosing protocol, comprising a) measuring blood lymphocytes and/or lymphocyte subsets in a first biological sample obtained from the patient during the induction phase, preferably wherein the lymphocyte subset is selected from the group comprising or consisting of CD4+ T cells, CD8+ T cells, CD19+ B cells, CD56+NK cells, and/or CD34+/CD45+ hematopoietic progenitor cells; b) measuring blood lymphocytes and/or one or more lymphocyte subsets in a second biological sample obtained from the individual during the chronic phase; c) determining whether there is a change in lymphocyte and/or lymphocyte subset levels between the first and second samples, and d) in the event that the total lymphocyte count and/or certain lymphocyte subset count(s) decrease between the induction phase and the chronic phase, reverting said patient to a SID schedule of 4-week intervals and/or a once-a-month dosing regimen, or increasing the dose frequency of the EID schedule, e.g. administering every five weeks instead of every six weeks. In some embodiments, the first biological sample is obtained from the patient at least 6 months after initiation of the induction phase, and the second biological sample is obtained within 6 months of the initiation of the chronic phase.

In some embodiments, a decrease in total lymphocyte count of >1×10⁹ cells indicates a potential reduction in treatment efficacy and that the patient's dosing schedule should be changed. In another embodiment, a decrease in CD4+ T cell count of at least about 300 cells/mm indicates a potential reduction in treatment efficacy and that the patient's dosing schedule should be changed. In another embodiment, a decrease in CD8+ T cell count of at least about 200 cells/mm³ cells indicates a potential reduction in treatment efficacy and that the patient's dosing schedule should be changed. In another embodiment, a decrease in CD19+ B cell count of at least about 300 cells/mm³ indicates a potential reduction in treatment efficacy and that the patient's dosing schedule should be changed. In another embodiment, a decrease in CD56+NK cell count of at least about 100 cells/mm³ indicates a potential reduction in treatment efficacy and that the patient's dosing schedule should be changed. In another embodiment, a decrease in CD34+/CD45+ progenitor cell count of at least about 5 cells/mm³ indicates a potential reduction in treatment efficacy and that the patient's dosing schedule should be changed.

As described herein, biphasic dosing regimens comprising SC administration are provided for increasing the safety of natalizumab therapy. In some embodiments, the biphasic dosing regimens are provided for increasing the safety of chronic natalizumab therapy. Safety may be increased by reducing the risk of an adverse event as compared to SID. As an exemplary embodiment, the biphasic regimen reduces the risk of PML. In some cases, the biphasic regimen reduces the risk of PML, reduces the risk of inducing generation of anti-natalizumab antibodies, reduces the risk of patient sensitization to natalizumab, or a combination thereof. In some cases, the biphasic regimen reduces the risk of loss of efficacy of natalizumab treatment due to the generation of anti-idiotypic antibodies to natalizumab in the patient.

In some embodiments, the risk of developing PML in a subject receiving natalizumab on a biphasic dosing regimen comprising SC administration described herein is reduced by at least 20% relative to the risk of developing PML in a subject receiving natalizumab therapy on a SID schedule, and the efficacy of the natalizumab therapy is reduced by less than 10% relative to the efficacy of SID. For example, the risk of developing PML in a subject receiving natalizumab on a biphasic dosing regimen comprising SC administration described herein is reduced by at least 30%, 40%, or 50% relative to the risk of developing PML in a subject receiving natalizumab therapy on a SID schedule of 4-week intervals, and the efficacy of the natalizumab therapy is reduced by less than 10% relative to the efficacy of SID.

A subject, as provided herein, is typically a male or female human subject (patient) who is undergoing or who will undergo treatment with natalizumab for a particular condition. The condition may be an autoimmune condition or an inflammatory condition. Often, autoimmune conditions are considered inflammatory conditions and vice versa, thus, in some embodiments the subject has an autoimmune condition and/or inflammatory condition. An autoimmune condition is a condition in which a subject's immune system attacks the subject's own cells/tissues. Non-limiting examples of autoimmune conditions include multiple sclerosis (MS) (e.g., relapsing-remitting MS, secondary progressive MS, and/or primary progressive MS), Crohn's disease, rheumatoid arthritis, lupus, celiac disease, Sjorgren's syndrome, Polymyalgia rheumatic, ankylosing spondylitis, Type 1 diabetes, alopecia areata, vasculitis, and temporal arteritis. Many of the foregoing conditions are also inflammatory conditions. Thus, in some embodiments, the methods of the present disclosure comprise identifying a subject for natalizumab therapy on a biphasic dosing regimen comprising SC administration, or administering to a subject natalizumab therapy on a biphasic dosing regimen comprising SC administration, wherein the subject is at high risk for PML and has an autoimmune condition. In some embodiments, the autoimmune condition is multiple sclerosis. In some embodiments, the autoimmune condition is Crohn's disease.

In some embodiments, the subject has been diagnosed with epilepsy. Epilepsy is a central nervous system disorder (neurological disorder) in which nerve cell activity in the brain becomes disrupted, causing seizures or periods of unusual behavior, sensations and sometimes loss of consciousness. Thus, in some embodiments, the methods of the present disclosure comprise identifying a subject for natalizumab therapy on a biphasic dosing regimen comprising SC administration, or administering to a subject natalizumab therapy on a biphasic dosing regimen comprising SC administration, wherein the subject has epilepsy, has recently had a seizure, or has epilepsy and has recently had a seizure. In some embodiments, the subject is at high risk for PML and has epilepsy, has recently had a seizure, or has epilepsy and has recently had a seizure.

In some embodiments, a subject has a prior history of immunosuppression. In some embodiments, a subject was treated with an immunosuppressant prior to receiving natalizumab therapy on SID schedule of 4-week intervals. In particular embodiments, the immunosuppressant is selected from the group comprising or consisting of mitoxantrone, methotrexate, mycophenlate mofetil, azathioprine, and cyclophosphamide.

A high PML risk subject is a subject who is seropositive for anti-JCV antibodies. In some embodiments, a high PML risk subject has had prior immunosuppression and is seropositive for anti-JCV antibodies. In some embodiments, a PML risk subject has an anti-JCV antibody index level (e.g., a mean index level) of greater than 1.5. In some embodiments, a low PML risk subject is a subject who has an anti-JCV antibody index level (e.g., a mean index level) of less than or equal to 0.9. Anti-JC virus index values are calculated from a two-step ELISA antibody assay of serum/plasma (STRATIFY JCV™ Antibody (with Index) with Reflex to Inhibition Assay; see, e.g., Lee, P. et al. J of Clin Virol, 2013; 57(2):141.146, incorporated herein by reference). Antibody index level, assays for assessing index level, and the use of such index levels and assays, for determining PML risk are described in, e.g., WO 2012/166971 and WO 2014/193804.

A subject may be considered a high PML risk if the subject tested seropositive for anti-JCV antibodies prior to commencement of natalizumab therapy, or if the subject switches from a seronegative anti-JCV antibody status to a seropositive anti-JCV antibody status during natalizumab therapy. In some embodiments, a subject is considered a high PML risk if the subject has an anti-JCV antibody index level of greater than 1.5 prior to commencement of natalizumab therapy, or if the subject switches from a lower anti-JCV antibody index level of less than or equal to 0.9 to a higher anti-JCV antibody index level of greater than 1.5 during natalizumab therapy. For example, prior to starting natalizumab therapy, a subject may be tested for the presence or absence of anti-JCV antibodies. If the test results indicate that the subject is a low PML risk subject (seronegative for anti-JCV antibodies, or having an anti-JCV antibody index level of less than or equal to 0.9), then the subject may be identified as a subject for natalizumab therapy on a SID schedule of 4-week intervals. During the course of the natalizumab therapy on a SID schedule, the subject may be re-tested for the presence or absence of anti-JCV antibodies (e.g., tested every month or every 2, 3, 4, 5 or 6 months, or every year). If upon re-testing the subject has switched from seronegative to seropositive for anti-JCV antibodies, or from having an anti-JCV antibody index level of less than or equal to 0.9 to having an anti-JCV antibody index level of greater than 1.5, then the subject may be identified as a subject for natalizumab therapy on an EID schedule of at least 5-week intervals.

Exemplary embodiments include a method of treating multiple sclerosis (MS) in a subject, comprising: a. administering to a subject a therapeutically effective amount of natalizumab on a SID schedule of 4-week intervals for at least 12 months, followed by chronically administering to the subject a therapeutically effective amount of natalizumab on an EID schedule of at least 6-week intervals; b. determining whether the natalizumab therapy on the EID schedule maintains a trough α4-integrin saturation of at least, or of at least about, 40%, 45%, 50%, 55%, 60%, 65% or 70%; and c. if the trough α4-integrin saturation has fallen below the designated level then reverting the patient back to the SID schedule, or increasing the dose frequency of the EID schedule, e.g. administering every five weeks instead of every six weeks.

Exemplary embodiments include a method of treating Crohn's disease in a subject, comprising: a. administering to a subject a therapeutically effective amount of natalizumab on a SID schedule of 4-week intervals for at least 12 months, followed by chronically administering to the subject a therapeutically effective amount of natalizumab on an EID schedule of at least 6-week intervals; b. determining whether the natalizumab therapy on the EID schedule maintains a trough α4-integrin saturation of at least, or of at least about, 40%, 45%, 50%, 55%, 60%, 65% or 70%; and c. if the trough α4-integrin saturation has fallen below the designated level then reverting the patient back to the SID schedule, or increasing the dose frequency of the EID schedule, e.g. administering every five weeks instead of every six weeks.

In some cases of one or more exemplary embodiments, step (b) comprises a) measuring a soluble molecule in a biological sample obtained from the patient during the SID schedule, wherein the soluble molecule is sVCAM; b) measuring the sVCAM in a second biological sample obtained from the individual during the EID schedule; c) determining whether there is an increase in the level of sVCAM between the first and second biological samples, wherein the increase correlates with a decrease in the trough α4-integrin saturation in the individual. In some embodiments, the first biological sample is obtained from the patient at least 6 months after initiation of the SID schedule, and the second biological sample is obtained within 6 months of the initiation of the EID schedule.

Although the foregoing biphasic dosing regimens are described in terms of natalizumab therapy, it is understood that such biphasic regimens are likely to be suitable for use with other α4-integrin binding antibodies that increase PML risk, and in particular those that bind the same epitope as, or compete for epitope binding with, natalizumab, or those that inhibit lymphocyte trafficking to the brain.

### EXAMPLES

### Example 1: Pharmacokinetics and Pharmacodynamics of Natalizumab Every 4 Weeks Following Subcutaneous and Intravenous Administration

Background: Natalizumab 300 mg infused intravenously (IV) every 4 weeks (Q4W) is an efficacious treatment for patients with relapsing forms of multiple sclerosis that works by binding to alpha-4 integrin on lymphocytes to prevent their migration from the vasculature into the central nervous system. Subcutaneous (SC) administration may be preferred by patients due to increased convenience and time savings compared with IV administration. SC natalizumab has been developed and studied in 2 clinical trials, DELIVER (NCT00559702) and REFINE (NCT01405820).

Objectives: To characterize the pharmacokinetic (PK)/pharmacodynamic (PD) (alpha-4 integrin saturation) profile of natalizumab after single and multiple doses of SC administration to support SC dose selection using population PK/PD model-based simulations to achieve efficacy similar to 300 mg IV.

Methods: In DELIVER, single-dose PK/PD was collected over 8 weeks, with sampling at 4 hours and on days 1, 2, 3, 4, 7, 14, 21, 28, 35, 42, and 56; multi-dose trough PK/PD was collected for an additional 6 doses over 24 weeks. A population PK/PD model was previously developed using clinical data from 11 clinical trials. Steady-state natalizumab trough serum concentrations and alpha-4 integrin saturations over time for natalizumab 300, 350, 400, and 450 mg SC Q4W were simulated for 1000 subjects per SC dose and compared with the PK/PD profile of natalizumab 300 mg IV Q4W.

Results: After a single SC dose, natalizumab concentration peaked at approximately 168 hours (7 days) and 4 hours with SC and IV administration, respectively, as seen in Table 1. The elimination phase was similar with SC and IV administration. Alpha-4 integrin saturation occurred rapidly, with greater than 80% saturation achieved within 4 hours. PD was similar with IV and SC dosing at all time points assessed. Over multiple doses, natalizumab trough concentrations and corresponding alpha-4 integrin saturation levels were similar with SC and IV dosing. The lowest SC trough concentration, observed with 300 mg dosing, was 22 mg/mL, which is greater than the complete PD response concentration (defined as a concentration resulting in 70% alpha-4 integrin binding).

TABLE 1
PK and PD of SC- and IV-administered natalizumab

| | PK | | PD | |
|---|---|---|---|---|
| Endpoint | SC (n = 22) | IV (n = 24) | SC (n = 22) | IV (n = 24) |
| Single-dose time to peak | 168 | 4 | 72 | 24 |
| natalizumab serum concentration or | | | | |
| alpha-4 integrin saturation (hours) | | | | |
| Single-dose peak natalizumab | 35.7 (10.4) | 108.2 (26.9) | 94.1 (5.8) | 96.2 (3.2) |
| concentration (μg/mL) or alpha-4 | | | | |
| integrin saturation (%), mean (std | | | | |
| dev) | | | | |
| Single-dose natalizumab | 2.9 (2.4) | 5.6 (6.3) | 46.1 (26.3) | 50.2 (26.5) |
| concentration (μg/mL) or alpha-4 | | | | |
| integrin saturation (%) at day 56, | | | | |
| mean (std dev) | | | | |
| Q4W dosing natalizumab | 27.2 (15.3) | 29.8 (19.5) | 81.3 (18.7) | 86.2 (7.2) |
| concentration or alpha-4 integrin | | | | |
| saturation (%) at steady state, mean | | | | |
| (std dev) | | | | |

Conclusions: Single- and multi-dose observed PD as well as simulations conducted using population PK/PD modeling indicate that 300 mg SC dosing results in similar alpha-4 integrin saturation as 300 mg IV dosing. These data support 300 mg SC dosing and predict that it will achieve efficacy similar to the currently approved 300 mg IV dosing.

### Example 2: Modeling and Simulation Comparison of Pharmacokinetics and Pharmacodynamics of Natalizumab Every 6 Weeks Following Intravenous and Subcutaneous Dosing

Background: Natalizumab 300 mg administered intravenously (IV) approximately every 6 weeks (Q6W) is associated with significantly lower risk of progressive multifocal leukoencephalopathy than natalizumab 300 mg IV dosing every 4 weeks (Q4W). Subcutaneous (SC) natalizumab has been developed and studied in 2 clinical trials, DELIVER (NCT00559702) and REFINE (NCT01405820), with the expectation that SC administration will offer greater convenience and time savings than IV administration. Clinical studies have suggested that 300 mg Q4W IV and SC have comparable pharmacodynamics (PD) and efficacy. No clinical trials have provided pharmacokinetic (PK)/PD or efficacy data for natalizumab SC Q6W.

Objectives: To utilize a preexisting natalizumab population PK/PD model to simulate PK/PD profiles of natalizumab SC Q6W and compare these simulations to predicted IV Q6W data and to interim observed PK/PD Q6W IV data from the NOVA clinical trial (NCT03689972).

Methods: Trough natalizumab concentration (PK) and alpha-4 integrin saturation (PD) following 300 mg Q6W IV and SC administration were simulated using a preexisting population PK/PD model. Final parameter estimates from the model and a virtual population of 1000 subjects with covariate distribution observed in previous clinical trials were used for simulations. PK/PD results for patients who switched to Q6W dosing after 1 year of Q4W dosing (10,000 simulated subjects) were compared with observed interim PK/PD results for patients on the same dosing regimen in NOVA.

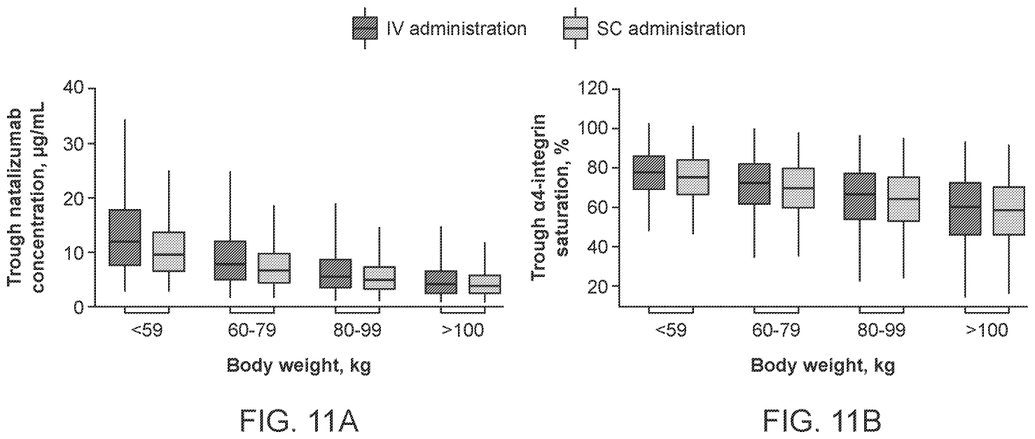

Results: Predicted median (95% prediction interval) steady-state natalizumab trough concentrations and alpha-4 integrin saturations for Q6W IV and SC dosing were comparable: 7.22 (0.61-24.76) mg/mL and 6.11 (0.58-18.39) mg/mL and 71.4% (15.8%-99.8%) and 68.6% (14.8%-97.8%), respectively. Median alpha-4 integrin saturation was ≥70% with both Q6W IV and SC dosing. Predicted steady state Q6W natalizumab concentration (PK) and alpha-4 integrin saturation (PD) each decreased in a similar fashion with increasing body weight (FIG. 11). Model-predicted PK and PD ranges following the switch to Q6W IV dosing encompass the observed data from NOVA.

Conclusions: These results predict that trough natalizumab concentration and alpha-4 integrin saturation will be similar for Q6W IV and SC dosing, suggesting that similar efficacy and safety profiles can be expected for IV and SC administration. The agreement of observed and predicted data for IV Q6W dosing bolsters confidence in the predictive capability of the model. PK/PD data for natalizumab Q6W SC from the planned NOVA open-label extension may provide further validation of the model.

### Example 3: Comparison of Switching to 6-Week Dosing of Natalizumab Versus Remaining on 4-Week Dosing in Patients with Relapsing-Remitting Multiple Sclerosis: A Randomized Controlled Study (NOVA)

Background: Patients with relapsing-remitting multiple sclerosis (RRMS) treated with natalizumab every-4-weeks (Q4W) who switch to extended interval dosing with an approximate dosing interval of every-6-weeks (Q6W) have significantly reduced risk of progressive multifocal leukoencephalopathy (PML) compared with patients on Q4W dosing. A randomized clinical trial was performed to assess the efficacy of natalizumab Q6W in patients with RRMS.

Objectives: To test the effectiveness of natalizumab EID and Q4W dosing in comparable patients who switch to EID (dosing frequency of approximately Q6W) after 1.2 years stable treatment on Q4W.

Methods: NOVA was a controlled, prospective, open-label, rater-blinded multinational study in patients from 89 sites treated with natalizumab 300 mg by intravenous infusion Q4W for ≥12 months who were randomized 1:1 to switch to natalizumab intravenous Q6W dosing or to continue Q4W treatment. The primary endpoint was the number of new/newly enlarging T2 (N/NET2) hyperintense lesions, by MRI at week 72 and was evaluated in the modified intent-to-treat population (all randomized patients who received 1 dose of study treatment and had 1 postbaseline efficacy assessment). Safety was evaluated all randomized patients who received 1 dose of study treatment. NOVA is registered with clinicaltrials.gov (NCT03689972).

Results: Between Dec. 26, 2018 and Aug. 30, 2019, 499 patients were randomly assigned across 89 study sites. Of these, 251 were randomly assigned to receive natalizumab Q6W, and 248 were assigned to continued treatment with natalizumab Q4W. Approximately equal proportions of Q6W (207 of 251 [82%]) and Q4W (195 of 248 [79%]) patients completed the study. Baseline demographic and disease characteristics of the Q6W (n=247) and Q4W (n=242) mITT populations were well balanced, including similar median MS disease duration (100 vs 90 years), median number of relapses in the prior year (10 for both), and median duration of natalizumab exposure prior to randomisation (4.0 years for both) (Table 2). Compliance up to the last dose was 10000 for Q6W and 99.9% for Q4W patients. Mean time between natalizumab doses was 6.0 (standard deviation [SD] 0.06)

TABLE 2
Demographic and disease characteristics at study baseline (mITT population)

| | Q6W (n = 247) | | Q4W (n = 242) | | Total (N = 489) | |
|---|---|---|---|---|---|---|
| Demographic Characteristic^{a} | | | | | | |
| Age, years | 40.9 | (9.66%) | 40.3 | (9.94%) | 40.6 | (9.80%) |
| Sex, female | 174 | (70.4%) | 176 | (72.7%) | 350 | (71.6%) |
| Ethnicity, not Hispanic or Latino | 220 | (89.1%) | 219 | (90.5%) | 439 | (89.8%) |
| Race | | | | | | |
| White | 208 | (84.2%) | 205 | (84.7%) | 413 | (84.5%) |
| Black or African American | 14 | (5.7%) | 23 | (9.5%) | 37 | (7.6%) |
| Asian | 4 | (1.6%) | 1 | (0.4%) | 5 | (1.0%) |
| American Indian or Alaska Native | 1 | (0.4%) | 1 | (0.4%) | 2 | (0.4%) |
| Other | 5 | (2.0%) | 1 | (0.4%) | 6 | (1.2%) |
| Not reported^{b} | 15 | (6.1%) | 11 | (4.5%) | 26 | (5.3%) |
| Region | | | | | | |
| North America^{c} | 129 | (52.2%) | 130 | (53.7%) | 259 | (53.0%) |
| Europe and Israel^{d} | 101 | (40.9%) | 98 | (40.5%) | 199 | (40.7%) |
| Australia | 12 | (4.9%) | 9 | (3.7%) | 21 | (4.3%) |
| United Kingdom | | | | | | |
| Height, mean (SD), cm | 169.10 | (8.83) | 168.36 | (9.43) | 168.74 | (9.13) |
| Weight, mean (SD), kg | 79.70 | (19.59) | 78.62 | (20.28) | 79.16 | (19.92) |
| ≤80 kg | 146 | (59.1%) | 138 | (57.0%) | 284 | (58.1%) |
| BMI, mean (SD), kg/m2 | 27.74 | (6.70) | 27.51 | (6.71) | 27.63 | (6.70) |
| Disease Characteristic^{e} | | | | | | |
| Time since MS symptoms onset, years^{f} | 10.0 | (6.0, 15.0)^{g} | 9.0 | (5.0, 15.0) | 10.0 | (6.0, 15.0)^{h} |
| Time since RRMS diagnosis, yearsⁱ | 8.0 | (4.0, 13.0)^{j} | 8.0 | (4.0, 12.0)^{k} | 8.0 | (4, 13.0)^{l} |
| Relapses during the previous 12 | 1.0 | (0.0, 2.0)^{k} | 1.0 | (0.0, 1.0)^{m} | 1.0 | (0.0, 1.0)ⁿ |
| months | | | | | | |
| Duration of natalizumab exposure at | 4.0 | (2.1, 6.6) | 4.0 | (2.2, 6.1) | 4.0 | (2.1, 6.5) |
| baseline, years | | | | | | |
| Patients with no missed doses in the 3 | 247 | (100%) | 241 | (99.6%) | 488 | (99.8%) |
| months prior to screening | | | | | | |
| Patients without dosing gap >3 months | 227 | (91.9%) | 229 | (94.6%) | 456 | (93.3%) |
| EDSS score at baseline, mean (SD) | 2.32 | (1.3) | 2.31 | (1.3) | 2.31 | (1.30) |
| T2 hyperintense lesion volume, mL | 10.0 | (4.8, 18.5) | 9.6 | (4.3, 18.2) | 9.9 | (4.6, 18.4) |
| T1 hypointense lesion volume, mL | 0.6 | (0.2, 1.7) | 0.6 | (0.1, 1.7) | 0.6 | (0.2, 1.7) |
| Normalised brain volume, mL | 1516.4 | (1453.4, 1572.7) | 1532.5 | (1459.1, 1579.0) | 1523.9 | (1456.1, 1577.2) |

^{a}Demographic characteristics are n (%) unless indicated otherwise;
^{b}not reported due to confidentiality regulations;
^{c}includes USA and Canada;
^{d}includes Belgium, France, Germany, Israel, Italy, Netherlands, and Spain;
^{e}disease characteristics are median (IQR) unless indicated otherwise;
^{f}calculated as date of randomization minus date of MS onset;
^{g}n = 246;
^{h}n = 488;
ⁱcalculated as date of randomization minus date of diagnosis;
^{j}n = 245;
^{k}n = 241;
^{l}n = 486;
^{m}n = 236;
ⁿn = 477.
BMI = body mass index; IQR = interquartile range; mITT = modified intent to treat; SD = standard deviation; Q4W = every 4 weeks; Q6W = every 6 weeks.

Intercurrent events (events leading to treatment discontinuation) were reported for 46 of 247 (18.6%) Q6W patients and 51 of 242 (21.1%) Q4W patients, and mean time to first intercurrent event was similar for the two arms (Q6W 33.1 [SD, 18.75] weeks; Q4W 30.3 [SD, 21.83] weeks). Most intercurrent events were not related to treatment (Q6W 35 of 46 [76.1%]; Q4W 44 of 51 [86.3%]) or had no information on the reason for the event (Q6W 4 of 46 [8.7%]; Q4W 4 of 51 [7.8%]). Intercurrent events due to INEC-confirmed relapse were reported for similar proportions in the 2 arms (Q6W: 7 of 247 [2.8%]; Q4W: 5 of 242 [2.1%]). However, the number of relapsed patients who opted for rescue treatment was imbalanced between the 2 arms: 6 of 7 Q6W patients chose optional rescue therapy after relapse while none of the Q4W patients did so.

The primary endpoint (number of new/newly enlarging T2 lesions at week 72) was 0.20 (95% CI: 0.07, 0.63) in the Q6W group and 0.05 (95% CI: 0.01, 0.31) in the Q4W group (ratio, 4.24 [95% CI: 0.86, 20.85]; p=0076) with the treatment policy strategy estimand and 0.31 (95% CI: 0.12, 0.82) in the Q6W group and 0.06 (95% CI: 0.01, 0.31) in the Q4W group (ratio, 4.93 [95% CI: 1.05, 23.20]; p=0.044) with the hypothetical strategy estimand (Table 3).

TABLE 3
Primary endpoint analysis: new/newly enlarging
T2 lesions at week 72 (mITT population)

| Patients with lesions | Q6W (n = 247) | | Q4W (n = 242) | |
|---|---|---|---|---|
| 0 | 202 | (81.8%) | 189 | (78.1%) |
| 1 | 5 | (2.0%) | 7 | (3.6%) |
| 2 | 2 | (0.8%) | 1 | (0.5%) |
| 3 | 0 | | 0 | |
| 4 | 0 | | 0 | |
| ≥5 | 2 | (0.8%)^{a} | 0 | |
| Missing | 36 | (14.6%) | 45 | (18.6%) |
| Number of lesions, mean (SD) | 0.3 | (2.69) | 0 | (0.23) |
| Range | 0, 30 | | 0, 2 | |
| Adjusted mean number of lesions (treatment | 0.20 | (0.07, 0.63) | 0.05 | (0.01, 0.22) |
| policy estimand), 95% CI^{b, c} | | | | |
| Ratio of adjusted mean lesion numbers, | 4.24 | (0.86, 20.85) | — | |
| Q6W:Q4W, 95% CI^{b, c} | | | | |
| p value^{b, c} | 0.076 | | — | |
| Adjusted mean number of lesions (hypothetical | 0.31 | (0.12, 0.82) | 0·06 | (0.01, 0.31) |
| strategy estimand), 95% CI^{b, d} | | | | |
| Ratio of adjusted mean lesion numbers, | 4.93 | (1.05, 23.20) | — | |
| Q6W:Q4W, 95% CI^{b, d} | | | | |

| | | |
|---|---|---|
| p value^{b, d} | 0.044 | — |

^{a}One patient had 30 lesions; one patient had 25 lesions.
^{b}Estimated by negative binomial regression with treatment as classification and baseline body weight (≤80 vs >80 kg), duration of natalizumab exposure at baseline (≤3 vs >3 years), and region (North America, United Kingdom, Europe and Israel, and Australia) as covariates.
^{c}Observed lesions are included for analysis, regardless of intercurrent events, and missing values due to efficacy or safety (2 switched to Q4W and 1 discontinued treatment for Q6W; 1 discontinued treatment for Q4W) are imputed by the worst case of patients on treatment at the same visit in the same treatment group; otherwise via multiple imputation.
^{d}Observed lesions before intercurrent events are included for analysis, and missing data due to efficacy (6 switched to Q4W, and 1 discontinued treatment for Q6W; 1 discontinued treatment for Q4W) are imputed by the worst case of patients on treatment; otherwise via multiple imputation.

The distributions of patients with 0 (78.1% vs. 81.8%), 1 (2.9% vs. 2.0%), and 2 (0.4% vs. 0.8%) new/newly enlarging hyperintense T2 lesions at week 72 were similar between the Q4W and Q6W dosing groups, respectively. However, two patients in the Q6W group had ≥5 lesions while no Q4W patient had >2. Of these two Q6W patients, Patient 1 had no new disease activity while on treatment but discontinued natalizumab at week 55 because of the development of anti-JCV seropositive status and experienced relapse at week 67. Subsequent MRI showed 30 new/newly enlarging T2 lesions. Since the T2 lesion increase in Patient 1 occurred off treatment, Patient 1 contributed to treatment policy strategy estimand only and the 30 lesion value was not imputed for other Q6W patients with missing data. Patient 2 was diagnosed with asymptomatic PML after completing 72 weeks of treatment. Regularly scheduled MRI scans showed the presence of five new/newly enlarging T2 lesions at week 24, ten additional lesions at week 48, and a further ten lesions at week 72 (25 lesions total). Since the T2 lesion increase in Patient 2 occurred on treatment, this patient contributed to the assessment of the primary endpoint under both the treatment policy strategy and hypothetical strategy estimands. Additionally, the week 72 T2 lesion value (25) was used as the worst value for on-treatment imputation for the six Q6W patients who opted for optional rescue treatment.

Secondary clinical outcomes were not significantly different for mITT patients randomized to natalizumab Q6W or Q4W dosing. The cumulative probability of relapse at 72 weeks was similar for Q6W and Q4W (0.97 vs 0.98) as was the estimated time to first relapse (hazard ratio [HR], 1.31 [95% CI: 042, 4.13]; p=0.64). ARR at 72 weeks was not significantly different for Q6W and Q4W patients (0.00013 [95% CL: 000006, 0.00027] vs 0.00010 [95% CI: 0.00004, 0.00024]; ratio, 1.32481 [95% CI (0.42016, 4.17725]; p=0.63). The cumulative probability of 24-week CDW at 72 weeks was similar in both groups (Q6W 0.90; Q4W 0.92), as was the time to 24-week CDW (HR, 1.29 [95% CI: 0.71, 2.34]; p=0.40).

Secondary MRI outcomes at all timepoints were similar for 247 Q6W and 242 Q4W mITT patients. At 72 weeks, 1 Q6W and 1 Q4W patients (0.4% each) had Gd+ lesions. The Q6W patient with ≥5 Gd+ lesions at week 72 was the same patient with 30 new/newly enlarging T2 lesions after treatment discontinuation (Patient 1, described above). At the same time point, 3 Q6W patients (I 2%) and 2 Q4W patients (0.8%) had new T1 hypointense lesions. One of the Q6W patients with T1 hypointense lesions was Patient 1 and other was the asymptomatic PML case with 25 new/newly enlarging T2 lesions (Patient 2, above). At 48 weeks, 7 Q6W patients (28%) and 6 Q4W patients (2.5%) had new/newly enlarging T2 lesions. In the Q6W arm, Patient 1 (30 lesions at week 72) and Patient 2 (25 lesions at week 72) had 0 and 15 lesions at week 48, respectively. While the prespecified analyses treated all lesions as MS, the etiology of the week 24 and 48 T2 lesions in Patient 2 is unknown.

The safety population included 250 Q6W and 247 Q4W patients. The incidence of AEs and SAEs were similar between the two treatment arms. Over the course of the study, SAEs were experienced by 17 of 250 (6.8%) Q6W patients and 17 of 247 (6.9%) Q4W patients. AEs leading to discontinuation of study treatment were reported by 4 of 250 (1.6%) Q6W patients and 1 of 247 (0.4%) Q4W patients. No deaths were reported in either arm.

One case of PML occurred in the natalizumab Q6W treatment arm (Patient 2, above), with no cases in the Q4W group. Patient 2 had known risk factors for PML, including total natalizumab exposure of 2 years (1 year prior to study enrolment and 1 year of on-study Q4W dosing) and an anti-JCV antibody index greater than the limit of assay detection (reported as >2.35) at enrolment and all subsequent assessments. The patient had not been treated with any DMT for the year prior to initiating natalizumab after seven years of injectable or oral DMT treatment. The case was classified as asymptomatic PML and is ongoing.

The exploratory NEDA endpoint was achieved by similar proportions of patients in both arms (Q6W 173 of 247 [70.0%]; Q4W 163 of 242 [67.4%]; OR 1.1 95% CI 0.8-1.7]; p=0.52).

As optional rescue was imbalanced between groups based on relapse criteria, an ad hoc analysis was performed to assess if differences in the frequency or severity of relapses could explain the imbalance. Proportions of patients meeting rescue criteria in the Q6W and Q4W groups who required steroid treatment (4 of 7 [57.1%] vs 4 of 5 [80.0%]) or hospitalization (1 of 7 [14.3%] vs 1 of 5 [20.0%]) were similar between groups, indicating that relapse events were of similar frequency and severity overall despite the observed imbalance in the proportions of patients taking rescue treatment.

Conclusions: The results of this study indicate that disease activity remains low in patients who switch to natalizumab Q6W after ≥1 year of stable Q4W treatment. The findings further suggest that a majority of patients stable on natalizumab Q4W dosing can switch to Q6W dosing with little or no clinically meaningful loss of efficacy. The safety profile of natalizumab Q6W dosing was similar to that of Q4W dosing, and no new safety signals were identified.

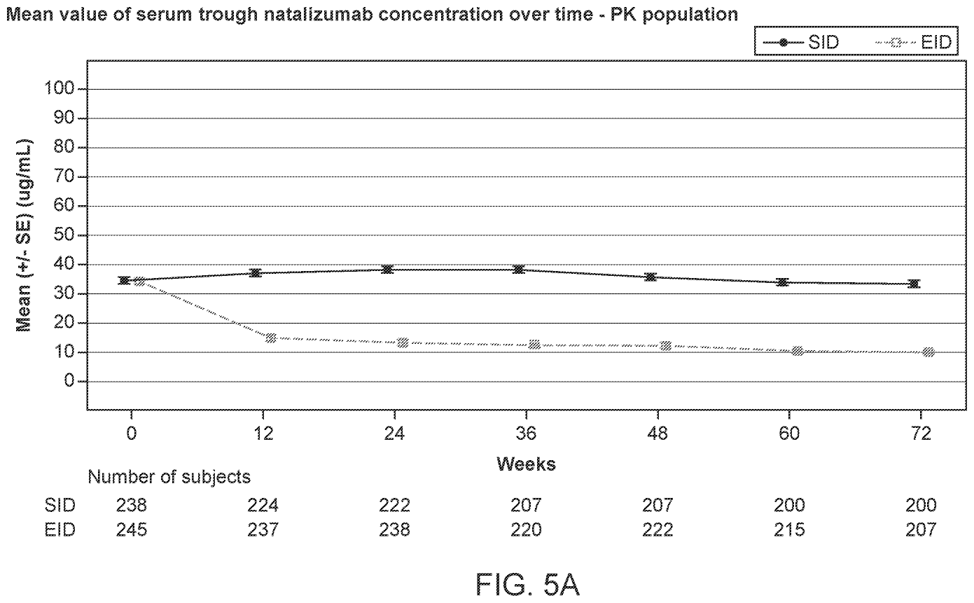

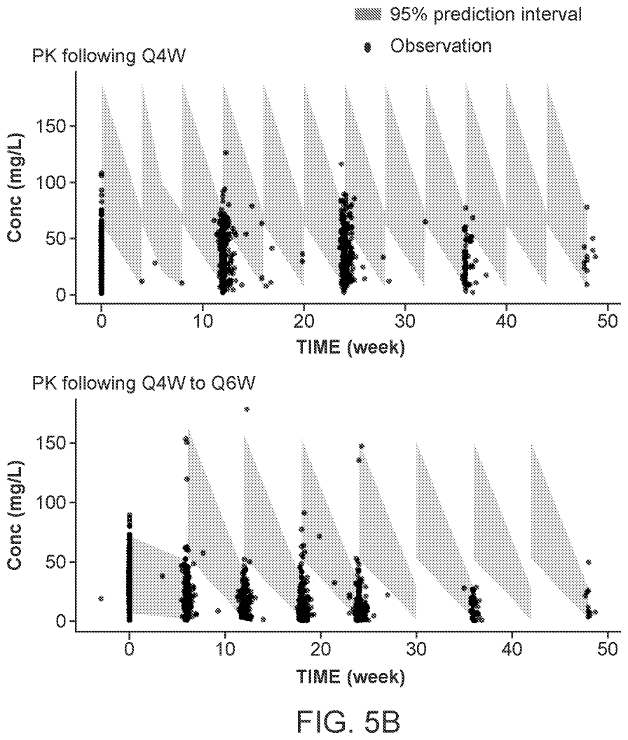

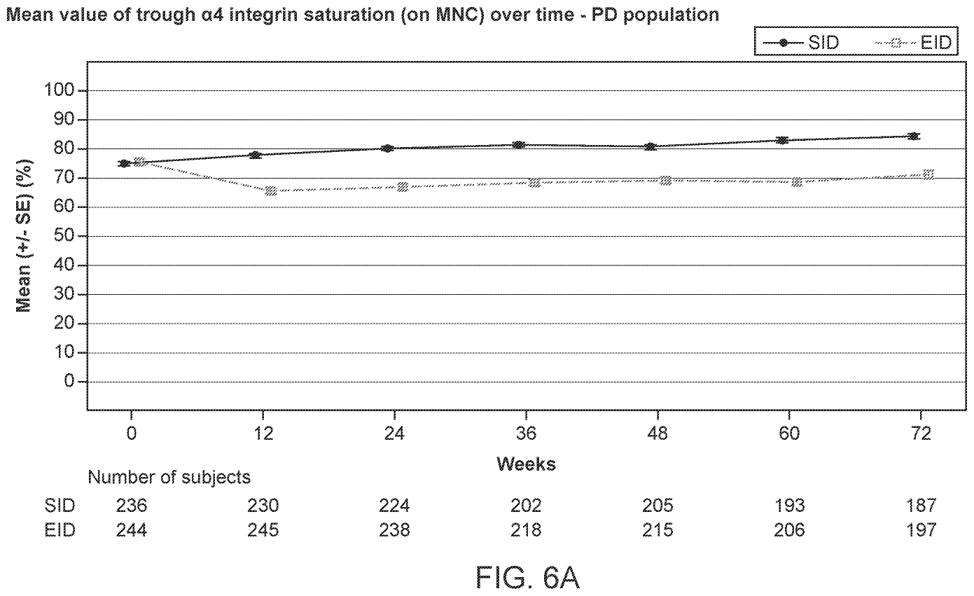

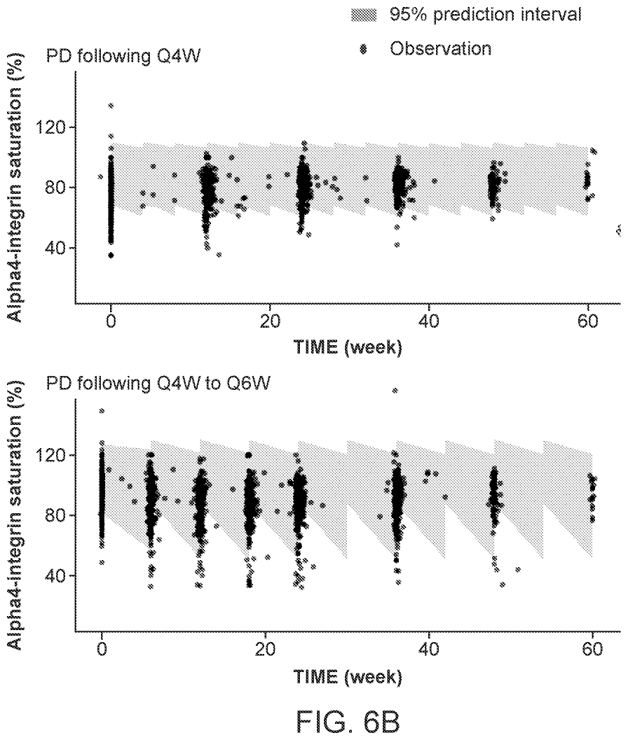

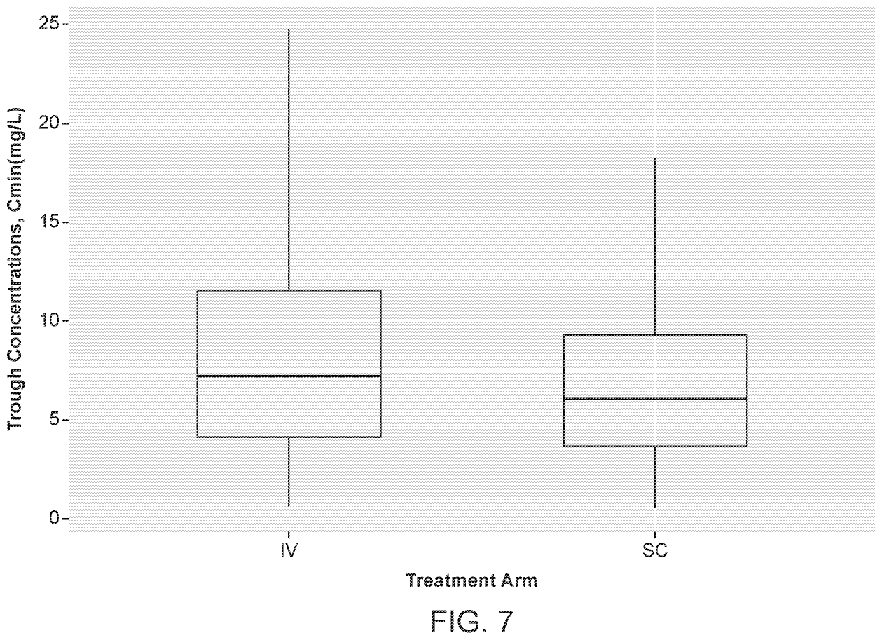

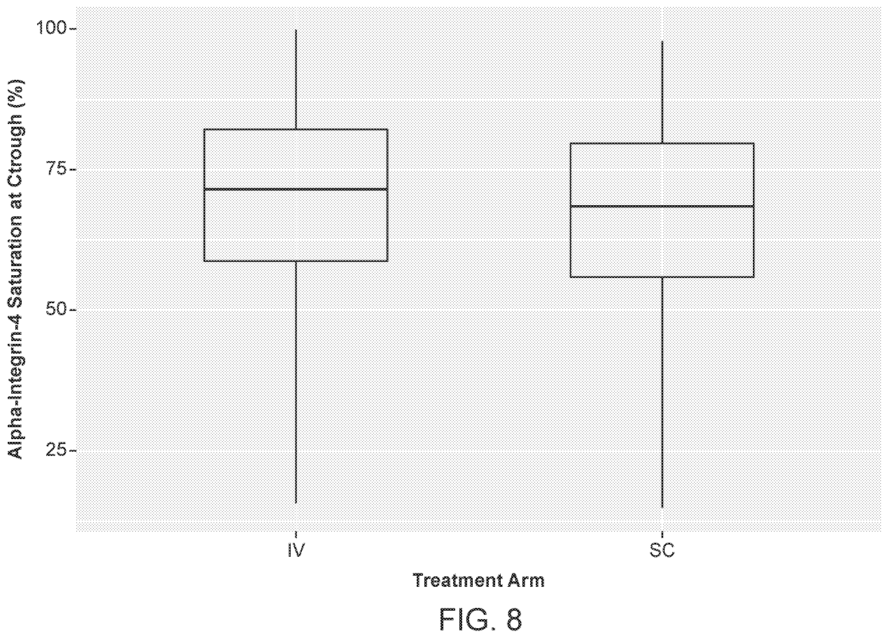

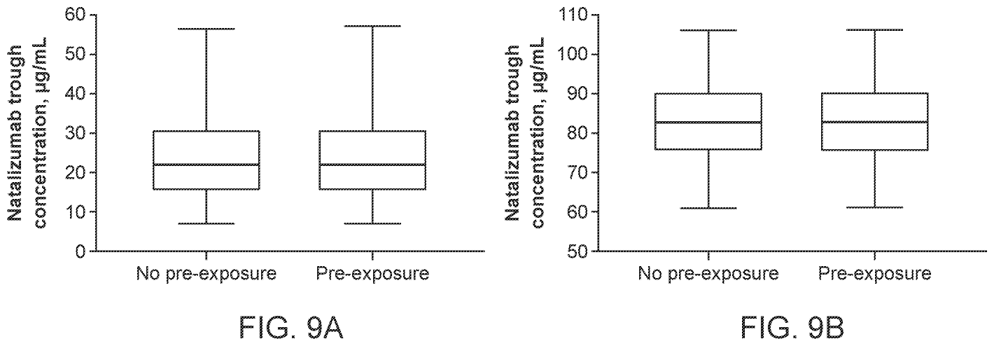

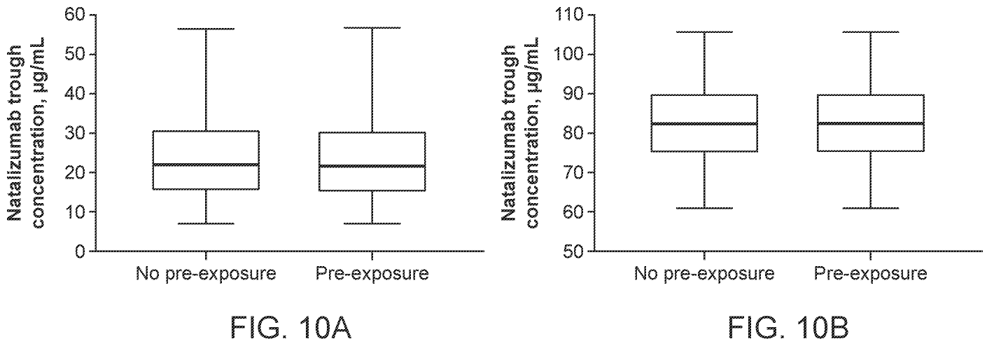

Pharmacokinetics and Pharmacodynamic Conclusions: Mean trough concentrations for the Q6W group was approximately 60% to 70% lower than for the Q4W group. Even with the lower PK exposure in the Q6W group compared to the Q4W group, α4 integrin saturation (on MNCs, monocytes, lymphocytes, B cells, T cells, and dendritic cell) was comparable between the Q6W and Q4W groups, with the difference in α4 integrin saturation ranging from 9% to 16%. (See, e.g., FIGS. 5 & 6)

### Example 4: Subject Preference for Subcutaneous (SC) Versus Intravenous (IV) Route of Natalizumab Administration

Background: Patients with relapsing-remitting multiple sclerosis (RRMS) who participated in the randomized controlled study (NOVA) disclosed in Example III, will participate in an Open-Label Crossover Extension (OLE) Study Comprising Subcutaneous and Intravenous.

Objectives: To evaluate subject preference for SC versus IV route of natalizumab administration and to and explore long-term efficacy, safety, and tolerability of EID.

Methods Subjects who participated in the study disclosed in Example 3, receive natalizumab 300 mg by IV infusion once every 6 weeks (42±7 days) for a period of 36 weeks and be randomized to an additional 48 weeks of crossover treatment comprising 24 weeks EID SC Q6W and 24 weeks EID IV Q6W. All MRI scans are read at a central facility by blinded raters. At the completion of their 48-week crossover treatment period, subjects receive a final dose of natalizumab 300 mg by SC injection or IV infusion at Week 156 with the route of administration being the subject's choice, proceed to the 12-week follow-up period, and receive a follow-up safety phone call 12 weeks later (i.e., 24 weeks after the last dose of study treatment) before completing the study. The primary endpoint is the proportion of subjects indicating a preference for natalizumab SC administration at the end of the study period.

Secondary endpoints are assessed between 6 months of SC treatment and 6 months of IV treatment in the randomized crossover period, and include: satisfaction with SC versus IV route of administration, comparison of drug preparation and administration time between SC and IV routes of natalizumab administration, evaluation of the safety and immunogenicity of SC versus IV routes of natalizumab administration, evaluation of the efficacy of SC versus IV routes of natalizumab administration, and analysis of the pharmacokinetics and pharmacodynamics of SC versus IV routes of natalizumab administration. Subjects who discontinue study treatment any reason will be withdrawn from the study and will be treated as per local standard of care.

All references, patents and patent applications disclosed herein are incorporated by reference in the entirety and for all purposes, and in particularly with respect to the subject matter for which each is cited, which in some cases may encompass the entirety of the document.

What is claimed is:

1. A method of reducing pathological inflammation in a patient in need thereof comprising administering a therapeutically effective amount of an anti-VLA-4 antibody to the patient in a biphasic dosing regimen, wherein the biphasic regimen comprises an induction phase comprising administration of the anti-VLA-4 antibody once a month for 10 to 14 months, preferably at least 12 months, followed by a chronic phase comprising administration of the anti-VLA-4 therapy once every 5, 6, 7 or 8 weeks, preferably wherein at least one phase of the biphasic protocol comprises subcutaneous (SC) administration.

2. A method of reducing progressive multifocal leukoencephalopathy (PML) in a patient known or suspected of suffering from multiple sclerosis, comprising administering a therapeutically effective amount of an anti-VLA-4 antibody to the patient in a biphasic dosing regimen, wherein the biphasic regimen comprises an induction phase comprising administration of the anti-VLA-4 antibody once a month for 10 to 14 months, preferably at least 12 months, followed by a chronic phase comprising administration of the anti-VLA-4 therapy once every 5, 6, 7 or 8 weeks, preferably wherein at least one phase of the biphasic protocol comprises subcutaneous (SC) administration.

3. The method of claim 1, wherein the pathological inflammation is caused by multiple sclerosis, and the therapeutically effective amount is sufficient to relieve symptoms of multiple sclerosis.

4. The method of any one of claims 1-3, wherein the anti-VLA-4 antibody is natalizumab.

5. The method of any one of claims 1-4, wherein administration during the induction phase is intravenous (IV) or subcutaneous (SC), preferably SC.

6. The method of any one of claims 1-5, wherein administration during the chronic phase is IV or SC, preferably SC.

7. The method of any one of claims 1-6, wherein both phases of the regimen comprise SC administration.

8. The method of any one of claims 1-7, wherein the therapeutically effective amount administered during the induction phase and the chronic phase are the same; preferably wherein the therapeutically effective amount is 300 mg.

9. The method according to any one of claims 1-7, wherein the therapeutically effective amount administered SC during the chronic phase is about 300 mg, about 350 mg, about 400 mg, about 450 mg, or about 500 mg, more preferably about 300 mg, still more preferably 300 mg.

10. A method of administering natalizumab to a patient in need thereof based on a biphasic dosing regimen, the method comprising administering the natalizumab therapy subcutaneously on an SID schedule for an induction phase of at least 12 months, and then administering the natalizumab therapy on an EID schedule of at least 6-week intervals chronically thereafter, wherein one or both, and preferably both, treatment phases comprise SC administration.

11. The method of claim 10, wherein the therapeutically effective amount administered during the induction phase and the chronic phase are the same; preferably wherein the therapeutically effective amount is 300 mg.

12. The method according to claim 9, wherein the therapeutically effective amount administered SC during the chronic phase is about 300 mg, about 350 mg, about 400 mg, about 450 mg, or about 500 mg, more preferably about 300 mg, still more preferably 300 mg.

13. The method according to any one of claims 1-12, further comprising a) measuring a soluble molecule in a first biological sample obtained from the patient during the induction phase, wherein the soluble molecule is serum vascular cell adhesion molecule (sVCAM) and/or neurofilament light chain (Nf-L); b) measuring the sVCAM and/or Nf-L in a second biological sample obtained from the individual during the chronic phase; c) determining whether there is an increase in the levels of the sVCAM and/or Nf-L above predetermined levels between the first and second biological samples, and d) in the event of an increase above said predetermined levels reverting said patient to an SID schedule or increasing the dose frequency of the EID schedule.

14. A method for determining and/or monitoring the efficacy of a biphasic dosing protocol for natalizumab having an induction phase comprising an SID schedule and a chronic phase comprising an EID schedule in a patient in need thereof, the method comprising a) measuring a soluble molecule in a first biological sample obtained from the patient during the induction phase, wherein the soluble molecule is sVCAM and/or Nf-L; b) measuring the sVCAM and/or Nf-L in a second biological sample obtained from the individual during the chronic phase; c) determining whether there is an increase in the levels of the sVCAM and/or Nf-L above predetermined thresholds between the first and second samples, and d) in the event of an increase above one or both predetermined thresholds reverting said patient to an SID schedule or increasing the dose frequency of the EID schedule.
